(19) United States
(12) Patent Application Publication
(10) Pub. No.: US 2025/0073454 A1
(43) Pub. Date: Mar. 6, 2025

(54) Set for Neurostimulation

(71) Applicant: PAJUNK GMBH MEDIZINTECHNOLOGIE, Geisingen (DE)

(72) Inventors: Simone PAJUNK-SCHELLING, Geisingen (DE); Martin HAUGER, Donaueschingen (DE)

(73) Assignee: PAJUNK GMBH MEDIZINTECHNOLOGIE, Geisingen (DE)

(21) Appl. No.: 18/821,911

(22) Filed: Aug. 30, 2024

(30) Foreign Application Priority Data
Sep. 5, 2023 (DE) 10 2023 123 897.3

(51) Int. Cl. A61N 1/05; A61M 5/34

(52) U.S. Cl. CPC A61N 1/0504; A61M 5/347; A61M 2205/054

(57) ABSTRACT

The application relates to a set for neurostimulation with a device for neurostimulation with a distal end, wherein the device comprises an electrically conductive stimulation element and a duct for the supply of a fluid, and with an indwelling cannula with an indwelling cannula conduit with a distal end and a proximal end, wherein the device comprises a first connector part and the indwelling cannula comprises a second connector part complementary to the first connector part, wherein, by connecting the first connector part and the second connector part, the device is fixable in an axial position in which the distal end of the device projects beyond the distal end of the indwelling cannula, wherein, in the connected state of the first connector part and the second connector part, the first connector part and the second connector part are connected with one another such the a circumferential first seal is formed.

### CROSS-REFERENCE TO RELATED APPLICATION

The present application claims priority to German Patent Application No. 10 2023 123 897.3, filed Sep. 5, 2023, the entirety of which is incorporated herein by reference.

### FIELD OF APPLICATION

The application relates to a set for neurostimulation.

### BACKGROUND

For the alleviation of pain or for relaxing muscle tensions and contractions it is known to transmit electrical current pulses, for example low-frequency alternating current at low voltage by means of electrodes placed onto the skin in order to transcutaneously stimulate nerves in this manner. However, the skin herein represents comparatively high contact resistance. Furthermore, as a rule, the stimulation takes place over a large area.

It is furthermore known to equip rigid cannulae which are pierced through the skin, in particular also guided through an indwelling cannula, with an electrical connection such that they are stimulatable in order to obtain information regarding the positioning of the tip of the cannula in order to draw conclusions in particular such as to whether or not the cannula is in contact on a nerve.

The application therefore addresses the problem of specifying an improved feasibility for neurostimulation which, in particular, is of versatile use and with which preferably a more direct stimulation is feasible.

### SUMMARY

The problem of the application is resolved through a set for neurostimulation with the characteristics of patent claim 1.

Advantageous physical forms and further developments of the application are specified in the dependent claims.

The set for neurostimulation comprises a device for neurostimulation with a distal end, wherein the device comprises an electrically conductive stimulation element and a duct for the supply of a fluid, and comprises furthermore an indwelling cannula with an indwelling cannula conduit with a distal end and a proximal end, wherein the device comprises a first connector part and the indwelling cannula comprises a second connector part complementary to the first connector part, wherein by connecting the first connector part and the second connector part the device is localizable in an axial position in which the distal end of the device projects beyond the distal end of the indwelling cannula, wherein, in the connected state of the first connector part and of the second connector part, the first connector part and the second connector part are connected with one another such that a circumferential first seal is formed.

The set according to the application is suitable for neurostimulation as well as also for the supply of a fluid, for example of a liquid such as an anesthetic agent. By way of the indwelling cannula, the device can be positioned in the body for longer time periods, and the device enables manifold applications. The indwelling cannula can be fabricated of a synthetic material, preferably a polyamide, and preferably be implemented such that it is electrically insulating. The indwelling cannula in particular has greater flexural rigidity than the part of the device for neurostimulation that has been introduced into the indwelling cannula whereby advancing the device through the indwelling cannula conduit is simplified. On the other hand, implementing the indwelling cannula conduit to be flexible is desirable so that even with long-term dwelling of the indwelling cannula in the body, the effect on the patient is as uncomfortable as is feasible.

Due to the circumferential first seal the volume between the device for neurostimulation and the indwelling cannula is impermeable such that there is the feasibility of introducing fluid into this volume. This can lead to the advantage that observation of the device for neurostimulation disposed in the indwelling cannula by means of ultrasound is improved over the entire length of the indwelling cannula.

The first seal is preferably developed by contact of a first contact face of the first connector part on a second contact face of the second connector part whereby additional components, such as for example a sealing ring, can be avoided.

According to an advantageous further development, the first connector part comprises a distal face on which a circumferential first projection projecting in the axial direction is disposed and furthermore the second connector part comprises a proximal face on which a circumferential second projection, projecting in the axial direction, is disposed, wherein the first contact face is disposed on the outer side of the first projection, and the second contact face is disposed on the inner side of the second projection or, alternatively, the first contact face is disposed on the inner side of the first projection and the second contact face is disposed on the outer side of the second projection. In this manner is obtained a circumferential abutment surface extending over a section in the axial direction between the first contact face and the second contact face across a section, which abutment surface enables the reliable formation of the first seal.

The first contact face preferably comprises a circumferential beading projection in the direction toward the second contact face and/or the second contact face comprises a circumferential beading projecting in the direction toward the first contact face, which beading effects in particular that the first projection and the second projection are in contact on one another under pretensioning whereby the sealing effect can be improved.

According to some embodiments, the second connector part encompasses the indwelling cannula at a distal end forming a sealing and widens out in the direction toward a proximal end such that, adjoining the proximal end of the second connector part, an annular clearance is formed between the second connector part and the introduced device and that the first connector part comprises a circular groove open in the direction toward a distal end into which, in the connected state of the first connector part and of the second connector part, the proximal end of the second connector part immerges Such disposition can enable the simple connecting. Through this physical form the sealing effect can be improved.

The first projection is preferably disposed on a bottom surface of the circular groove. Through this physical form the seal formed between the first connector part and the second connector part can be disposed such that it is protected.

The duct of the device, in particular the tubing of the device, advantageously comprises a lateral opening which, in particular in the connected state of the first connector part and the second connector part, terminates into the annular clearance. This opening enables in simple manner filling fluid into the annular clearance between the second connector part as well as the indwelling cannula adjoining thereon, on the one hand, and filling fluid into the inserted device on the other.

According to some embodiments, connecting the first connector part and the second connector part takes place in the manner of a bayonet fitting, wherein preferably the latching of the bayonet fitting is haptically and/or acoustically perceptible. A bayonet fitting can be simply and rapidly mechanically opened and closed thereby that the two connector parts are inserted one into the other and turned in opposite directions. In the closed position a pin of the bayonet fitting latches into a lock reception. The resistance of the latching can preferably be selected of such magnitude that it is haptically and/or acoustically perceptible to a user, for example through a clicking sound, whereby the security of the operation can be increased.

The stimulation element is preferably developed as an electrically conductive stimulation wire with a diameter, a distal end and a proximal end, and the duct is preferably developed as an electrically insulating tubing with an inner diameter, an outer diameter, a distal end and a proximal end, wherein the stimulation wire is disposed in the tubing, wherein the inner diameter of the tubing is greater than the diameter of the stimulation wire such that an interspace for the supply of fluid is formed in the tubing, wherein at the distal end of the stimulation wire a stimulation electrode is disposed such that it is electrically conducting which projects beyond the distal end of the tubing. Thereby that the stimulation wire is disposed within the lumen of the tubing an especially compact structure can result.

Due to the flexibility of the tubing, moreover, an elongated flexible device can be provided which can be inserted either through cannulae or indwelling cannulae into the body but also through a natural body orifice, for example the nose, the bladder or the vagina, into the body without having to make an incision in the skin. Thereby also stimulation of even those nerves can take place which are only with difficulty or not at all accessible by stimulation electrodes adhering on the skin on the outside of the body.

The proximal end of the stimulation wire preferably projects beyond the proximal end of the tubing, and, after being bent around the proximal end of the tubing, is led to the outer side of the tubing, wherein an electrically conductive stimulation cable is connected at the proximal end of the stimulation wire such that it is electrically conducting and wherein an injection tube is slipped in sections over the proximal end of the tubing forming a seal. The stimulation wire, bent around at the proximal end of the stimulation wire, can enable a simple linkage of the stimulation cable on the outer side of the tubing without having to lead the stimulation wire through the wall of the tubing such that a possible leakage site can be avoided.

According to an advantageous further development of the application, the stimulation electrode comprises a rounded outer surface and is for example developed dome-shaped, spherical, ellipsoidal, mushroom-shaped, pear-shaped or egg-shaped.

Due to the rounded physical form, injuries on the body of the person to be treated can be avoided. Moreover, such physical form of the stimulation electrode can simplify advancing the device, in particular, into the body orifice.

The stimulation electrode preferably has in each cross section an outer diameter which is less than or equal to the outer diameter of the tubing. Stated differently, the outer dimensions of the stimulation electrode are not greater than the outer dimensions of the tubing such that the stimulation electrode does not project laterally. Such physical form can simplify advancing the device through the indwelling cannula.

It is fundamentally conceivable that fluid can escape at the distal end of the tubing between the stimulation electrode and the tubing wall. The stimulation electrode is advantageously disposed with a securement section under form closure in the distal end of the tube, wherein the tube comprises in its wall at least one injection opening terminating into the interspace between the stimulation wire and the tube and/or the stimulation electrode comprises an axial through-opening. The form-closure disposition enables the stabilization of the distal end of the stimulation wire within the tube. The disposition under form closure can furthermore enable a closure of the distal end of the tube in order to prevent the penetration of particles into the distal end of the tubing or even prevent clogging of the distal end of the tubing. In this physical form the fluid can exit laterally through the at least one injection opening disposed in the wall of the tubing and/or through the axial through-opening of the stimulation electrode.

The proximal end of the tube with the connection site to the stimulation cable and the connection site to the injection tube are preferably disposed in a housing, wherein the stimulation cable and the injection tube exit the housing proximally, in particular parallel to the longitudinal axis of the tubing and the first connector part is disposed on the housing and is connected in particular unitarily therewith. The proximal end of the tubing including the connection sites can herein be inlaid into the housing and optionally additionally be encast with an adhesive agent or be ensheathed with an injection molded housing. In this way the connection sites can be disposed under protection.

The diameter of the stimulation wire is preferably less than 0.3 mm, preferably less than 0.2 mm, especially preferably less than 0.1 mm. In this way a thin flexible stimulation wire can be provided which can enable the simple advance into the body.

The stimulation wire is preferably fabricated of special steel. The requisite stability of the stimulation wire can thereby be achieved in particular also at lengths of more than 10 cm.

According to some embodiments, the outer diameter of the tubing is less than 1.0 mm, preferably less than 0.9 mm. Thereby dimensioning of the device for neurostimulation can be enabled which can enable the simple introduction into the body.

The inner diameter of the tubing is preferably in the range of 0.4 mm to 0.7 mm, preferably in the range of 0.5 mm to 0.6 mm. Such dimensioning enables a sufficiently large interspace between the inner wall of the tubing and the stimulation wire in order to be able to guide a fluid through the tubing from the proximal up to the distal end.

According to some embodiments, the length of the tubing is more than 5 cm, preferably more than 8 cm, especially preferred more than 10 cm. Thereby sufficient length is provided in order to be able to advance the device for neurostimulation sufficiently far into the body.

According to some embodiments, the tubing may be fabricated of a synthetic material, for example of a polyamide. Such a material can enable sufficient impermeability, high flexibility as well as good biocompatibility.

According to some embodiments, the set may comprise additionally a rigid cannula with a cannula conduit with a distal end and a proximal end for insertion into a body, onto which the indwelling cannula can be slid before the insertion and which at its proximal end comprises a third connector part, complementary to the second connector part, wherein, by connecting the third connector part and the second connector part, the cannula can be localized in an axial position in which the distal end of the cannula conduit projects beyond the distal end of the indwelling cannula, wherein the third connector part and the second connector part are connected with one another such that a circumferential second seal is formed. The rigid cannula enables the insertion of the indwelling cannula through the skin into the body in order to be able to also stimulate nerves which are not accessible by stimulation through the skin.

The second seal is advantageously formed by placing a third contact face of the third connector part on the second contact face of the second connector part, whereby additional components, such as for example a sealing ring, can be avoided.

According to some embodiments, the third connector part has a distal face on which a circumferential third projection projecting in the axial direction is disposed, and the third contact face is disposed on the outer side of the third projection and the second contact face is disposed on the inner side of the second projection or, alternatively, the third contact face is disposed on the inner side of the third projection and the second contact face is disposed on the outer side of the second projection. In this way, a circumferential abutment surface between the third contact face and the second contact face results extending across a section extending in the axial direction, which abutment surface enables reliable formation of the second seal.

The third contact face preferably comprises a circumferential beading projecting in the direction toward the second contact face and/or the second contact face comprises a circumferential beading projecting in the direction toward the third contact face, which, in particular, has the effect that the third projection and the second projection are in contact on one another under pretensioning whereby the sealing effect can be improved.

The third connector part advantageously comprises a circular groove open in the direction toward a distal end into which groove in the connected state of the third connector part and of the second connector part, the proximal end of the second connector part immerges. Such disposition can enable simple connecting. Moreover, through this physical form the sealing effect can be improved.

The third projection is preferably disposed on a bottom surface of the circular groove. Through this physical form the seal formed between the third connector part and the second connector part can be disposed such that it is protected against external factors.

The cannula conduit preferably comprises a lateral opening which, in particular in the connected state of the third connector part and of the second connector part, terminates into the annular clearance. This opening enables in simple manner the filling with fluid of the annular clearance between the second connector part as well as that of the indwelling cannula conduit adjoining thereon, on the one hand, and the introduced cannula on the other hand. Thereby, in particular when advancing the cannula into the body, the ultrasound visibility of the cannula can be improved.

According to an advantageous further development, connecting the third connector part and the second connector part takes place in the manner of a bayonet fitting, wherein preferably the latching of the bayonet fitting is haptically and/or acoustically perceptible. A bayonet fitting can be mechanically opened and closed simply and rapidly, thereby that the two connector parts are inserted one into the other and turned in opposite directions. In the closed position a pin of the bayonet fitting latches into a lock reception. The resistance of the latching can preferably be selected of such magnitude that it is haptically and/or acoustically perceptible to a user, for example through a clicking sound whereby the security of the operation can be increased.

It is especially preferable for the third connector part to be developed substantially identically to the first connector part.

The cannula conduit is advantageously fabricated of an electrically conductive material and connected with an electrically conductive stimulation cable on the outer side of the cannula conduit such that it is electrically conducting, wherein an injection tube is slipped in sections over the proximal end of the cannula conduit forming a seal. The cannula therewith is suitable for nerve stimulation as well as also for the supply of a fluid, for example a liquid such as an anesthetic agent, and can simultaneously be structured compactly.

The proximal end of the cannula conduit with the connection site to the stimulation cable and the connection site to the injection tube is preferably disposed in a housing, wherein the stimulation cable and the injection tube exit the housing proximally, in particular parallel to the longitudinal axis of the cannula tube, and the third connector part is disposed on the housing, in particular is connected with it unitarily. The proximal end of the injection tube including the connection sites can herein be inlaid into the housing and optionally additionally be encast with an adhesive agent or be ensheathed with an injection molded housing. In this way the connection sites can be disposed under protection.

Various embodiments will be explained in greater detail in conjunction with the following Figures. Therein depict

### BRIEF DESCRIPTION OF THE DRAWINGS

FIG. 1a a side view of a set for neurostimulation with a device for neurostimulation with a first connector part and an indwelling cannula with a second connector part, wherein the device is disposed in the indwelling cannula,

FIG. 1b the device for neurostimulation according to FIG. 1a without indwelling cannula,

FIG. 2 a longitudinal section through the device according to FIG. 1b,

FIG. 3 an enlargement of a cutout from FIG. 2 for the depiction of the electrical linkage of the stimulation wire of the device for neurostimulation,

FIG. 4 an enlargement of a cutout of the distal end of the device from FIG. 2,

FIG. 5 an enlargement of a cutout from FIG. 3 for the depiction of a bent-around section of the stimulation wire,

FIG. 6 a cutout of a longitudinal section through the device disposed in the indwelling cannula according to FIG. 2,

FIG. 7 an enlargement of a cutout from FIG. 6 with the first connector part and the second connector part connected,

FIG. 8 a view comparable to FIG. 7, wherein the first connector part and the second connector part are disconnected,

FIG. 9 a further enlargement of a cutout from FIG. 6 for the depiction of a lateral opening of the tubing of the device for neurostimulation,

FIG. 10 a longitudinal section through the indwelling cannula from FIG. 2 in which a cannula with a third connector part is disposed,

FIG. 11 an enlargement of a cutout from FIG. 10 with connected third connector part and second connector part,

FIG. 12 a view comparable to FIG. 11, wherein the third connector part and the second connector part are disconnected,

FIG. 13 an enlargement of a cutout from FIG. 10 for the depiction of the electrical linkage of the cannula,

FIG. 14 a further enlargement of a cutout from FIG. 10 for the depiction of a lateral opening in the cannula conduit of the cannula and

FIG. 15 a plan view onto the distal ends of the device for neurostimulation according to FIG. 1b and the cannula according to FIG. 10.

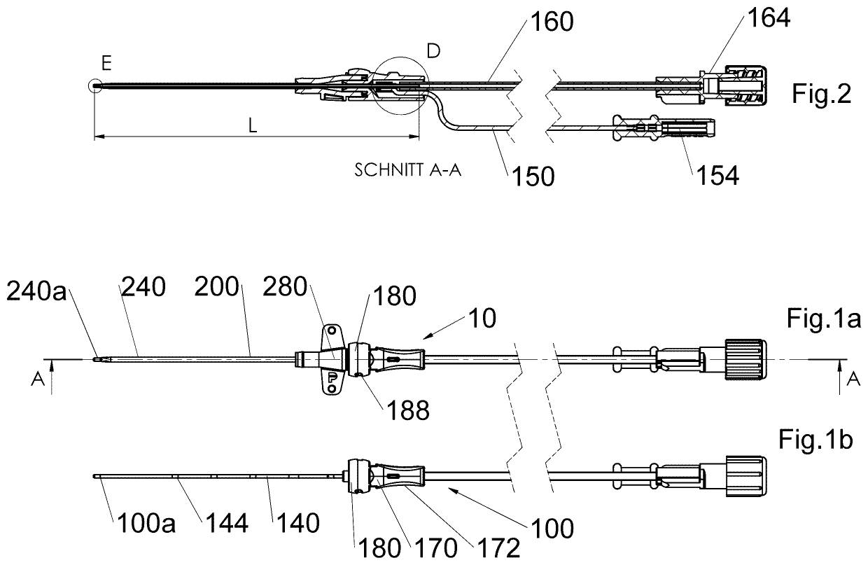

FIGS. 1 to 15 show various views of an embodiment example of a set 10 for neurostimulation. For greater clarity not all reference symbols are specified in all Figures.

### DETAILED DESCRIPTION

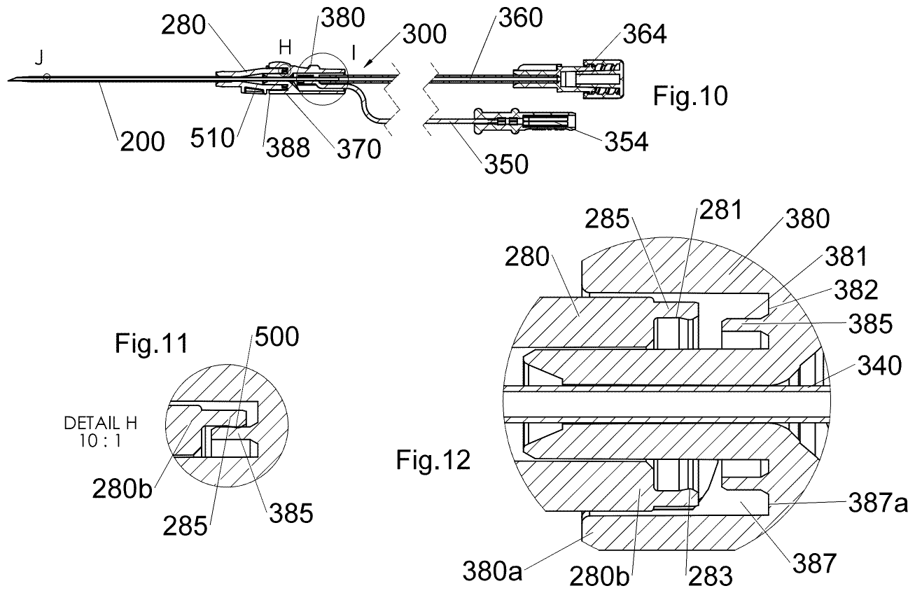

The set 10 for neurostimulation comprises at least one device 100 for neurostimulation and an indwelling cannula 200, which will be explained in greater detail in conjunction with FIGS. 1 to 9, and can optionally comprise additionally a cannula 300, which will be explained in greater detail in conjunction with FIGS. 10 to 15.

The device 100 comprises an electrically conductive stimulation element which, in particular, is developed as an electrically conductive stimulation wire 120 with a diameter AD, a distal end 120a and a proximal end 120b. The stimulation wire 120 can involve a flexible bendable wire. The stimulation wire 120 is fabricated, for example, of special steel. The diameter AD of the stimulation wire 20 can be less than 0.3 mm, preferably less than 0.2 mm, especially preferably less than 0.1 mm, for example approximately 0.07 mm.

The device 100 comprises furthermore a duct for the supply of a fluid, which is, in particular, developed as an electrically insulating tubing 140 with an inner diameter IS, an outer diameter AS, a distal end 140a and a proximal end 140b. The tubing 140 involves in particular a flexible, impermeable and biocompatible tubing 140. The tubing 140 can be fabricated, for example, of a synthetic material, especially of a polyamide. The outer diameter AS of tubing 140 is preferably less than 1.0 mm, in particular preferably less than 0.9 mm, and can be for example 0.87 mm. The inner diameter IS of tubing 140 is greater than the diameter AD of stimulation wire 120 and can be in the range of 0.4 mm to 0.7 mm, preferably in the range of 0.5 mm to 0.6 mm and be for example 0.54 mm. The length L of the tubing 140 is more than 5 cm, preferably more than 8 cm, for example 8.35 cm, and especially preferably more than 10 cm, for example 11.35 or 16.35 cm. Starting at the distal end 140a, the tubing 140 can comprise markings 144 (see FIG. 1b) which are disposed in particular equidistantly and can provide information to the user regarding the length of the distal end 140a of the tubing 140 that has been inserted into a body.

The stimulation wire 120 in particular is disposed in the tubing 140, in particular is guided through the lumen of tubing 140. Thereby that the inner diameter IS of the tubing 140 is greater than the diameter AD of the stimulation wire 120, an interspace 146 is formed in the tubing 140 through which, as will be described in greater detail in the following, fluid can be conducted from the proximal end 140b in the tubing 140 to the distal end 140a of the tubing 140. The interspace 146 can be developed in the manner of an annular clearance between the stimulation wire 120 and the inner wall of the tubing 140 when the stimulation wire 120 is disposed substantially centrally in the lumen. The stimulation wire 120 can alternatively also be in contact on the inner wall of tubing 140 such that the interspace 146 can be developed in the shape of a crescent.

At the distal end 120a of the stimulation wire 120, a stimulation electrode 130 is disposed, such that it is electrically conducting, which projects beyond the distal end 140a of tubing 140. The stimulation electrode 130 can have a rounded outer surface 132 and be developed, for example, dome-shaped, spherical, ellipsoidal, mushroom-shaped, pear-shaped or egg-shaped or in any other spheroidally rounded form. Due to the convex physical form of the stimulation electrode 130, it has, at least in cross section, a greater outer diameter AE. This outer diameter AE of the stimulation electrode 130, however, is at most as large as the outer diameter AS of tubing 140 (cf. in particular FIG. 4).

The electrically conducting connection between the stimulation wire 120 and the stimulation electrode 130 can be developed for example thereby that the distal end 120a of the stimulation wire 120 is fixed on a securement section 134 disposed on the stimulation electrode 130 by welding or clamping between the securement section 134 and the tubing 140 (see FIG. 4).

The stimulation electrode 130 with the securement section 134 can be disposed in the distal end 140a of the tubing 140, stated more precisely, in a section 148 of the tubing 140 extending from the distal end 140a, under form closure. The securement section 134 can comprise on its outer side one or more grooves which improve the clamping fixation of the securement section 134 in the section 148 of tubing 140. The stimulation electrode 134, in particular the securement section 134, can thereby substantially fill out the distal end 140a of tubing 140. In order to enable the fluid disposed within the interspace 146 to emerge at the distal end 100a of the device 100, the tubing 140 can comprise in its wall at least one injection opening (not depicted) terminating into the interspace 146 between the stimulation wire 120 and the tubing 140, and/or the stimulation electrode 130 can comprise an axial through-opening 136.

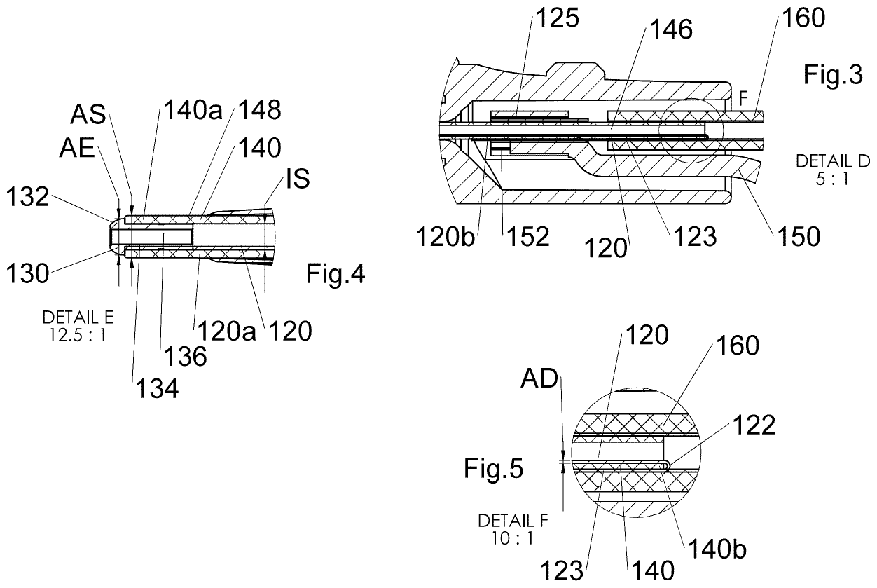

The proximal end 120b of the stimulation wire 120 projects beyond the proximal end 140b of tubing 140 and in in particular is bent in a bent-around section 122, in particular by 180°, around the proximal end 140b of tubing 140 and lead to the outer side of the tubing 140 (cf. FIGS. 3 and 5). An electrically conductive stimulation cable 150, in particular an electrically conductive lead 152 of the stimulation cable 150, is connected electrically conducting at the proximal end 120b of the stimulation wire 120 which, in particular, with a carried-back section 123 adjoining the bent-around section 122, comes to lie on the outer side of tubing 140. The electrically conducting contacting of the stimulation wire 120 can consequently take place outside the lumen which, when in use, is often fluid-filled, of tubing 140 and without breakthrough through the wall of tubing 140. The stimulation cable 150, in particular the lead 152 of the stimulation cable 150, can be connected electrically conducting, for example by means of a crimp sleeve 125, with the stimulation wire 120, in particular, the carried-back section 123 (see in particular FIG. 3). As is in particular discernible in FIG. 2 at the other end of the stimulation cable 150 an electric plug connection 154 can be disposed to connect a control unit with pulse generator.

An injection tube 160 can in sections be slipped over the proximal end 140b of tubing 140, and therein, in particular in sections, also be slipped over the carried-back section 123, located on the outer side of tubing 140, of the stimulation wire 120, forming a seal. This can be attained for example thereby that an inner diameter of the injection tube 160 corresponds approximately to the outer diameter AS of tubing 140. As is in particular discernible in FIG. 2, at the other end of the injection tube 160 an injection connection 164 can be disposed across which, for example with a syringe, a fluid, for example an anesthetic agent, can be injected into the injection tube 160 which through the injection tube 160 enters into the proximal end 140b of tubing 140 and exits at the distal end 140a of tubing 140, in particular through the axial through-opening 136 of the stimulation electrode 130.

The proximal end 140b of tubing 140, with the connection site to the stimulation cable 150 and the connection site to the injection tube 160, can be disposed in a housing 170, wherein the stimulation cable 150 and the injection tube 160 are led out of the housing 170 proximally, in particular parallel to the longitudinal axis of tubing 140. The proximal end 140b of tubing 140 including the connection sites can herein be inlaid into the housing 170 and additionally be encast with an adhesive agent or, alternatively, be ensheathed with an injection molded housing 170. In this way the connection sites can be disposed such that they are protected. On the housing 170 can be disposed one or several recessed grips 172 for placing a user's fingers in order to increase the grip certainty.

The indwelling cannula 200 comprises an indwelling cannula conduit 240 with an inner diameter IV, an outer diameter AV, a distal end 240a, and a proximal end 240b. The indwelling cannula conduit 240 can be fabricated of a synthetic material, preferably of a polyurethane, and preferably be fabricated of an electrically insulating material. Into the indwelling cannula conduit 240 can especially advantageously be integrated X-ray contrast strips. The inner diameter IV is greater than the outer diameter AS of tubing 140 of the device 100 so that the insertion of tubing 140 of the device 100 into the indwelling cannula conduit 240 is feasible. The indwelling cannula conduit 240 comprises in particular a higher flexural rigidity than the tubing 140.

For relative position fixation, the device 100 comprises a first connector part 180, while the indwelling cannula 200 comprises a second connector part 280, complementary to the first connector part 180, wherein, by connecting the first connector part 180 and the second connector part 280, the device 100 can be localized in an axial position in which the distal end 100a of the device 100 projects beyond the distal end 240a of the indwelling cannula 240. The first connector part 180 can be disposed in the proximity of the proximal end 140b of the tubing 140 of device 100 while the second connector part 280 can be disposed in the proximity of the proximal end 240b of the indwelling cannula conduit 240 of the indwelling cannula 100. In particular the first connector part 180 can be disposed on the housing 170 of device 100 and preferably be unitarily connected therewith.

In the connected state of the first connector part 180 and the second connector part 280, the first connector part 180 and the second connector part 280 are connected with one another such that a circumferential first seal 400 is formed (cf. FIG. 7). The first seal 400 can herein be developed, for example, by a first contact face 181 of the first connector part 180 coming to lie in contact on a second contact face 281 of the second connector part 280.

The first connector part 180 can comprise a distal face 182 on which a first projection 185 projecting in the axial direction is disposed. The second connector part 280 can comprise a proximal face 282 on which a circumferential second projection 285 projecting in the axial direction is disposed. As depicted in FIGS. 7 and 8, the first contact face 181 is disposed on the outer side of the first projection 185 and the second contact face 281 is disposed on the inner side of the second projection 285. In an alternative implementation, not depicted, it is also conceivable that the first contact face 181 is disposed on the inner side of the first projection 185 and the second contact face 281 is disposed on the outer side of the second projection 285. The two projections 185, 285 in both embodiments are disposed with respect to one another such that their contact faces 181, 182 slide past one another when the two connector parts 180, 280 are being connected.

As is discernible in particular in FIG. 8, the second contact face 281 can comprise a circumferential beading 283 projecting in the direction toward the first contact face 181. Alternatively, or additionally, the first contact face 181 can comprise a circumferential beading 283 projecting in the direction toward the second contact face 281 (not depicted).

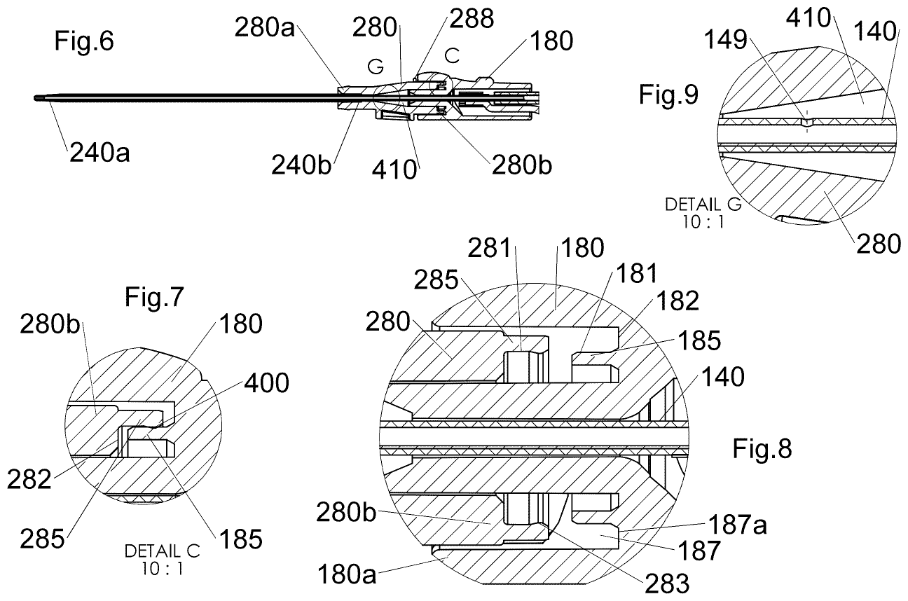

The second connector part 280 can encompass at a distal end 280a the indwelling cannula conduit 240, in particular the proximal end 240b of the indwelling cannula conduit 240, forming a seal, and can widen out in the direction toward a proximal end 280b. When the device 100 is inserted into the indwelling cannula 200 adjoining the proximal end 280b of the second connector part 280, an annular clearance 410 is thereby developed between the second connector part 280 and the inserted device 100 (cf. in particular FIGS. 6 and 9). The annular clearance 410 tapers in particular in the distal direction (cf. FIG. 9).

The duct of device 100, in particular the tubing 140 of the device 100, can comprise a lateral opening 149 which, in particular in the connected state of the first connector part 180 and the second connector part 280, terminates into the annular clearance 410.

The first connector part 180 can comprise a circular groove 187, open in the direction toward a distal end 180a of the first connector part 180, into which, in the connected state of the first connector part 180 and of the second connector part 280, immerges the proximal end 280b of the second connector part 280 (cf. in particular FIGS. 7 and 8). The first projection 185 can herein be disposed on a bottom surface 187a of the circular groove 187.

Connecting the first connector part 180 and the second connector part 280 can take place in the manner of a bayonet fitting, wherein, in particular for joining them, the proximal end 280b of the second connector part 280 is slid into the circular groove 187 of the first connector part 180 and subsequently the two connector parts 180, 280 are turned in opposite directions with respect to one another. A pin 288 of the second connector part 280 can herein be guided in a groove 188 of the first connector part 180. The latching of the bayonet fitting, in particular of the pin 288 at the end of groove 188 into a corresponding recess, is preferably haptically and/or acoustically perceptible.

The set 10 can furthermore comprise a rigid cannula 300 with a cannula conduit 340 with a distal end 340a and a proximal end 340b as well as an outer diameter AK and an inner diameter IK, wherein the distal end 340a comprises in particular a tip for insertion into a body (see in particular FIG. 15). The cannula tube 340 can be fabricated of an electrically conductive material, for example of special steel, and, apart from the distal end 340a and a linkage site to a stimulation cable 360, can be coated on the outer side such that it is insulating. The outer diameter AK of the cannula conduit 340 is less than the inner diameter IV of the indwelling cannula conduit 240 such that before the penetration the indwelling cannula conduit 240 can be slid onto the cannula conduit 340. On the outer side of the cannula conduit 340 ultrasound visibility-enhancing embossing can be applied.

The outer diameter AK of the cannula conduit 340 is preferably less than 1.0 mm, and can be, for example, 0.95 mm. The inner diameter IK of the cannula conduit 340 can be in the range of 0.4 mm to 0.7 mm and be, for example, 0.65 mm.

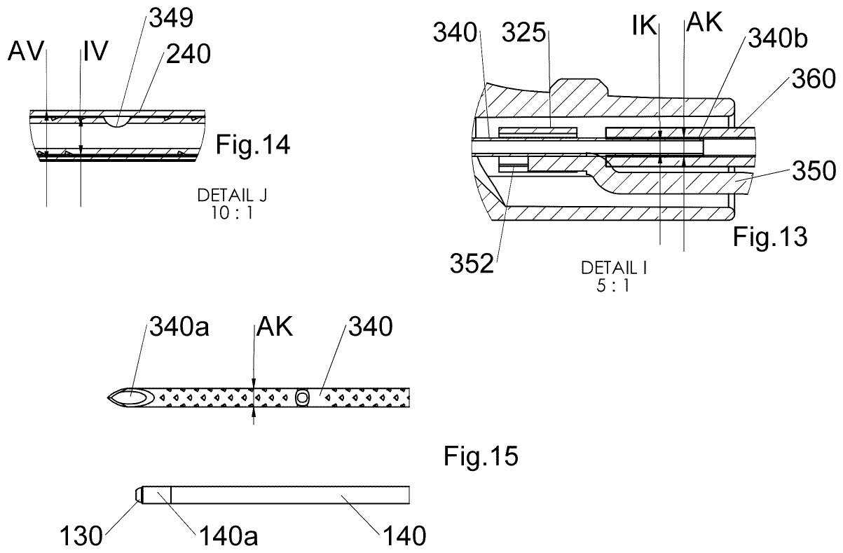

The electrically conductive contacting of the cannula conduit 340 can take place directly on the outer wall of the cannula conduit 340. For this purpose, a stimulation cable 350, in particular a lead 352 of the stimulation cable 350, is connected, for example by means of a crimp sleeve 325, with the cannula conduit 340 such that it is conducting (see in particular FIG. 13). As is discernible in particular in FIG. 10 at the other end of the stimulation cable 350 an electric plug connection 354 can be disposed for the connection of a control unit with pulse generator.

An injection tube 360 can in sections be slipped over the proximal end 340b of the cannula conduit 340 forming a seal (see FIG. 13). This can be attained for example thereby that an inner diameter of the injection tube 360 corresponds approximately to the outer diameter AK of the cannula conduit 340. As is in particular discernible in FIG. 10, at the other end of the injection tube 360 an injection connection 364 can be disposed across which, for example with a syringe, a fluid, for example an anesthetic agent, can be injected into the injection tube 360, which through the injection tube 360 enters into the proximal end 340b of the cannula conduit 340 and emerges at the distal end 340a of the cannula conduit 340, in particular through the sharpened tip.

The proximal end 340b of the cannula conduit 340 with the connection site to the stimulation cable 350 and the connection site to the injection tube 360 can be disposed in a housing 370, wherein the stimulation cable 350 and the injection tube 360 lead out of the housing 370 proximally, in particular parallel to the longitudinal axis of the cannula conduit 340 (see FIG. 13). The proximal end 340b of the cannula conduit 340 including the connection sites can herein be inlaid into the housing 370 and additionally be encast with an adhesive agent or, alternatively, be ensheathed with an injection molded housing. In this way the connection sites can be disposed so as to be protected. On the housing 370 one or more recessed grips 72 can be disposed for placing a user's fingers in order to increase the grip certainty.

The cannula 300 comprises a third connector part 380 which is developed complementarily to the second connector part 280 of the indwelling cannula 200 and enables the relative positioning of the cannula 300 and of the indwelling cannula 200. By connecting the third connector part 380 and the second connector part 280, the cannula 300 is localizable in an axial position in which the distal end 340a of the cannula conduit 340 projects beyond the distal end 240a of the indwelling cannula conduit 240. The third connector part 380 can be disposed in proximity to the proximal end 340b of the cannula conduit 340 of cannula 300. The third connector part 380 can in particular be disposed on the housing 370 of the cannula 300 and preferably be connected unitarily therewith.

In its functional features the third connector part 380 can correspond substantially to the first connector part 180. Thereby in particular the cannula 300 as well as also alternatively the device 100 for neurostimulation are fixed in the indwelling cannula 200 as will be explained in the following.

In the connected state of the third connector part 380 and of the second connector part 280, the third connector part 380 and the second connector part 280 are connected with one another such that a circumferential second seal 500 is developed (cf. FIG. 11). The second seal 500 can herein be developed, for example thereby that a third contact face 381 of the third connector part 380 is in contact on the second contact face 281 of the second connector part 280.

The third connector part 380 can comprise a distal face 382 on which a circumferential third projection 385 projecting in the axial direction is disposed. As is depicted in FIGS. 11 and 12, the third contact face 381 is disposed on the outer side of the third projection 385 and the second contact face 281 is disposed on the inner side of the second projection 285. In an alternative implementation, not shown, it is also conceivable that the third contact face 381 is disposed on the inner side of the third projection 385 and the second contact face 281 is disposed on the outer side of the second projection 285. In both embodiments the two projections 385, 285 are disposed relative to one another in particular such that their contact faces 281, 381 slide past one another when the two connector parts 280, 380 are being connected.

As is in particular discernible in FIG. 12 and already explained in conjunction with FIG. 8, the second contact face 281 can comprise the circumferential beading 283 projecting in the direction toward the second contact face 381. Alternatively, or additionally, the third contact face 381 can comprise a circumferential beading 283 projecting in the direction toward the second contact face 281 (not depicted).

As has already been explained, the second connector part 280 can encompass at a distal end 280a the indwelling cannula conduit 240, in particular the proximal end 240b of the indwelling cannula conduit 240 forming a seal and widening out in the direction toward the proximal end 280b. Even when the cannula 300 has been inserted into the indwelling cannula 200 thereby, adjoining the proximal end 280b of the second connector part 280, an annular clearance 510 is developed between the second connector part 280 and the inserted cannula 300 (cf. in particular FIG. 10). The annular clearance 510 tapers in particular in the distal direction and can, in particular, extend along the indwelling cannula conduit 240 up to its distal end 240a.

The cannula conduit 340 can comprise a lateral opening 349 which, in particular in the connected state of the third connector part 380 and of the second connector part 280, can terminate into the annular clearance 510 (cf. FIGS. 10 and 14).

The third connector part 380 can comprise a circular groove 387, open in the direction toward a distal end 380a of the third connector part 380, into which, in the connected state of the third connector part 380 and of the second connector part 280, the proximal end 280b of the second connector part 280 immerges (cf. in particular FIGS. 11 and 12). The third projection 385 can herein be disposed on a bottom surface 387a of the circular groove 387.

Connecting the third connector part 380 and the second connector part 280 can take place in the manner of a bayonet fitting wherein, in particular for the connection, the proximal end 280b of the second connector part 280, is slid into the circular groove 387 of the third connector part 380 and subsequently the two connector parts 380, 280 are turned in opposite directions relative to one another. Herein the pin 288 of the second connector part 280 can be guided in a groove 388 of the third connector part 380. The latching of the bayonet fitting, in particular of the pin 288 at the end of the groove 188 into a corresponding recess, is preferably haptically and/or acoustically perceptible. The set 10 can be utilized as follows.

Initially, the indwelling cannula 200 can be slid over the cannula conduit 340 of the cannula 300 and the second connector part 280 can be connected with the third connector part 380. The pointed distal end 340a of the cannula conduit 340 herein projects beyond the distal end 240a of the indwelling cannula conduit 240. These two components can jointly be pushed into a body. On the one hand, across the injection tube 360 a fluid can be injected into the cannula conduit 340, as well as through the lateral opening 349, into the annular clearance 510 between the cannula conduit 340 and the inner wall of the indwelling cannula conduit 240, which fluid improves the ultrasound visibility of the cannula conduit 340 when being inserting into the body, which however, due to the second seal 500 cannot escape into the body. On the other hand, across the stimulation cable 350, stimulation can take place thereby that current pulses are applied to the cannula conduit 340 in order to obtain information regarding the positioning of the tip of the cannula conduit 340 in the body.

If the cannula 300 is in the desired position, the connection between the third connector part 380 and the second connector part 280 can be disconnected and the cannula 300 can be withdrawn from the indwelling cannula 200 while the indwelling cannula 200 remains in its position in the body. The tubing 140 with the stimulation wire 120 of the device 100 for neurostimulation can subsequently be advanced through the indwelling cannula 200 until the distal end 100a of the device 100, in particular the stimulation electrode 130, is led out of the distal end 240a of the indwelling cannula 240. At this point stimulation by applying appropriate current pulses across the stimulation cable 150 can be carried out as well as also the injection of fluid, for example of an anesthetic agent, through the injection tube 160, can be carried out. The flexible indwelling cannula 200 also enables herein the long-term dwelling in the body as well as the positioning of the device 100 in the proximity of nerves otherwise only accessible with difficulty via the body surface.

### LIST OF REFERENCE SYMBOLS

10 Set
100 Device
100a Distal end
120 Stimulation wire
120a Distal end
120b Proximal end
122 Bend-around section
123 Carried-back section
125 Crimp sleeve
130 Stimulation electrode
132 Outer surface
134 Securement section
136 Axial through-opening
140 Tubing
140a Distal end
140b Proximal end
144 Marking
146 Interspace
148 Section
149 Lateral opening
150 Stimulation cable
152 Lead
154 Electric plug connection
160 Injection tube
164 Injection connection
170 Housing
172 Recessed grips
180 First connector part
180a Distal end
181 First contact face
182 Distal face
185 First projection
187 Circular groove
187a Bottom surface
188 Groove
200 Indwelling cannula
240 Indwelling cannula conduit
240a Distal end
240b Proximal end
281 Second contact face
282 Proximal face
283 Beading
285 Second projection
288 Pin
300 Cannula
325 Crimp sleeve
340 Cannula conduit
340a Distal end
340b Proximal end
349 Lateral opening
350 Stimulation cable
352 Lead
354 Electric plug connection
360 Injection tube
364 Injection connection
370 Housing
380 Third connector part
380a Distal end
381 Third contact face
382 Distal face
385 Third projection
387 Circular groove
387a Bottom surface [see above]
388 Groove
400 First seal
410 Annular clearance
500 Second seal
510 Annular clearance
AD Diameter of stimulation wire
AS Outer diameter of tubing
IS Inner diameter of tubing
AE Outer diameter of stimulation electrode
AV Outer diameter of indwelling cannula conduit [see above]
IV Inner diameter of indwelling cannula conduit
AK Outer diameter of cannula conduit
IK Inner diameter of cannula conduit
L Length of the tubing

1. A set for neurostimulation, comprising:
a device for neurostimulation with a distal end, wherein the device comprises:
an electrically conductive stimulation element,
a duct for supplying a fluid,
an indwelling cannula with an indwelling cannula conduit with a distal end and a proximal end, and
a first connector part,
wherein the indwelling cannula comprises a second connector part complementary to the first connector part,
wherein by connecting the first connector part and the second connector part the device is fixable in an axial position in which the distal end of the device projects beyond the distal end of the indwelling cannula,
wherein in a connected state of the first connector part and the second connector part, the first connector part and the second connector part are connected with one another such the a circumferential first seal is formed.

2. The set as in claim 1, wherein the first seal is formed by the contact of a first contact face of the first connector part on a second contact face of the second connector part.

3. The set in claim 2, wherein the first connector part comprises a distal face on which a circumferential first projection projecting in the axial direction is disposed, and the second connector part comprises a proximal face on which a circumferential second projection projecting in the axial direction is disposed, and the first contact face is disposed on the outer side of the first projection and the second contact face is disposed on the inner side of the second projection or, alternatively, the first contact face is disposed on the inner side of the first projection and the second contact face is disposed on the outer side of the second projection.

4. The set as in claim 2, wherein the first contact face comprises a circumferential beading projecting in the direction toward the second contact face or the second contact face comprises a circumferential beading projecting in the direction toward the first contact face.

5. The set as in claim 1, wherein the second connector part encompasses the indwelling cannula conduit at a distal end forming a seal and widens out in the direction toward a proximal end such that, adjoining the proximal end of the second connector part, an annular clearance is formed between the second connector part and the inserted device and that the first connector part comprises an annular groove, open in the direction toward a distal end, into which the proximal end of the second connector part immerges when the first connector part and the second connector part are in the connected state.

6. The set as in claim 5, wherein the first projection is disposed on a bottom surface of the annular clearance.

7. The set as in claim 5, wherein the duct of the device comprises a lateral opening which terminates into the annular clearance when the first connector part and the second connector part are in the connected state.

8. The set as in claim 1, wherein the connecting of the first connector part and the second connector part takes place in the manner of a bayonet fitting, wherein the latching of the bayonet fitting is haptically and acoustically perceptible.

9. The set as in claim 1, wherein the stimulation element is developed as an electrically conductive stimulation wire with a diameter (AD), a distal end and a proximal end, and the duct is developed as an electrically insulating tubing with an inner diameter (IS), an outer diameter (AS), a distal end and a proximal end, wherein the stimulation wire is disposed in the tubing, wherein the inner diameter (IS) of the tubing is greater than the diameter (AD) of the stimulation wire so that an interspace for the supply of fluid is formed in the tubing, wherein at the distal end of the stimulation wire a stimulation electrode is disposed such that it is electrically conducting, and which projects beyond the distal end of the tubing.

10. The set as in claim 9, wherein the proximal end of the tubing with the connection site to the stimulation cable and the connection site to the injection tube is disposed in a housing wherein the stimulation cable and the injection tube are led proximally and parallel to the longitudinal axis of the tubing out of the housing and the first connector part is disposed on and unitarily connected with the housing.

11. The set as in claim 1, wherein the set further comprises a rigid cannula with a cannula conduit with a distal end and a proximal end for insertion into a body, onto which the indwelling cannula can be slid before the insertion and which comprises at its proximal end a third connector part complementary to the second connector part, wherein, by connecting the third connector part and the second connector part, the cannula is fixable in an axial position in which the distal end of the cannula conduit projects beyond the distal end of the indwelling cannula, wherein the third connector part and the second connector part are connected with one another such that a circumferential second seal is formed.

12. The set as in claim 11, wherein the second seal is developed by the contact of a third contact face of the third connector part on the second contact face of the second connector part.

13. The set as in claim 12, wherein that the third connector part comprises a distal face on which is disposed a circumferential third projection projecting in the axial direction and the third contact face is disposed on the outer side of the third projection and the second contact face is disposed on the inner side of the second projection or, alternatively, the third contact face is disposed on the inner side of the third projection and the second contact face is disposed on the outer side of the second projection.

14. The set as in claim 12, wherein the third contact face comprises a circumferential beading projecting in the direction toward the second contact face and the second contact face comprises a circumferential beading projecting in the direction toward the third contact face.

15. The set as in claim 11, wherein the third connector part comprises an annular groove open in the direction toward a distal end into which, in the connected state of the third connector part and the second connector part, the proximal end of the second connector part immerges.

16. The set as in claim 15, wherein the third projection is disposed on a bottom surface of the circular groove.

17. The set as in claim 15, wherein the cannula conduit comprises a lateral opening which, in the connected state of the third connector part and the second connector part, terminates into the annular clearance.

18. The set a in claim 11, wherein the connecting of the third connector part and the second connector part takes place in the manner of a bayonet fitting, wherein the latching of the bayonet fitting is haptically and acoustically perceptible.

19. The set as in claim 11, wherein the cannula conduit is fabricated of an electrically conductive material and an electrically conductive stimulation cable is connected on the outer side of the cannula conduit such that it is electrically conductive and wherein an injection tube is slipped section wise over the proximal end of the cannula conduit forming a seal.

20. The set as in claim 19, wherein the proximal end of the cannula conduit with the connection site to the stimulation cable and the connection site to the injection tube is disposed in a housing, wherein the stimulation cable and the injection tube are led proximally and parallel to the longitudinal axis of the cannula conduit, out of the housing and the third connector part is disposed on and connected with the housing.
